(19) United States
(12) Patent Application Publication
(10) Pub. No.: US 2008/0037702 A1
(43) Pub. Date: Feb. 14, 2008

(54) Real-Time Navigational Aid System for Radiography

(76) Inventors: Jean-Noel Vallee, Paris (FR); Christophe Nioche, Paris (FR); Patrick Sabbah, Papeete (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/510,306

(22) PCT Filed: Apr. 4, 2003
(86) PCT No.: PCT/FR03/01075

§ 371 Date: Mar. 12, 2007

(30) Foreign Application Priority Data
Apr. 5, 2002 (FR) 02/04296

(51) Int. Cl. A61B 6/00

(52) U.S. Cl. 378/29; 600/407

(57) ABSTRACT

The invention provides a navigation method, located in the region of interest, that is designed to be used within a radiography unit including an X-ray source, recording systems placed in front of the source, and a support for the object to be radiographied. The method includes the following steps:
a) acquisition of tridimensional data on volume V1 images in the region of interest;
b) calculation, at time t, of a bidimensional projection image representing all or part of volume V1 and/or sub-volume of volume V1 depending on the position of the support, of the source and recording means, of a field of vision (FOV), focal distance (DF) and object distance (DO);
c) possible superposition or subtraction or fusion of the projection image and/or the sub-volume according to a given plane section of a radioscopy associated with the positions of the support, of the source and recording means, of a field of vision (FOV), focal distance (DF) and object distance (DO), at time t; and,
d) display of an image and/or a volume resulting from step c), and/or the projection image and/or the sub-volume.

The present invention concerns a guidance aiding system supported by imaging, for instruments and equipment inside a region of interest.

A volume in the region of interest is an object volume with no limitation of representation regarding the external or internal form of the object, obtained from any imaging technique capable of producing such volumes.

Real time active images are real time animated images obtained from any imaging technique capable of producing these images.

Instruments and equipment are instrumentations which can be visualized with the imaging technique that produces the real time active images.

At present, interventional radiology procedures assisted by angiography imaging, for example, for the investigation or the treatment of an anatomic region of interest, are presently carried out with a radio-guidance aiding system based on plane reference images according to 2 possible modes of radioscopy:
Radioscopy in superposition mode consists in superposing a plane reference image, subtracted or not, with inverted contrast or non-inverted contrast, previously acquired and stored, on the radioscopic image with the possibility of changing the mixing percentage of the reference image.
Radioscopy in so-called “road-map’ mode is a subtracted radioscopy. A subtracted plane image is generated during the radioscopy for use as a mask for the following radioscopies. In the vascular domain, an injection of contrast substance during the generation of the subtracted image produces a vascular cartography used as a reference mask for the following radioscopies. The opacified image of the vessels is subtracted from the active radioscopic image with a possibility to mix in variable proportion an anatomic background with the subtracted image.

Radioscopies in superposition mode and so-called “road-map” mode provide radio-guiding assistance according to the plane of the reference image, i.e. the projection plane of the image determined by the position of the cradle, the position of the anatomic region of interest, which depends from the position of the examination table, and according to the enlargement and scale of the reference image, which depend on the value of the field of vision and the geometric enlargement determined by the relation between the focal distance of the X-ray source to the recording system and the distance separating the X-ray source and the radiographied object. These modes of radioscopy have several disadvantages.

Firstly, for any change in the plane of the reference image, in the position of the anatomical region of interest, in the image enlargement or scale, the operator must either acquire and store a new reference image in the case of superposition mode radioscopy, or generate a new subtracted reference image in the case of road-map radioscopy. These iterative procedures result in lengthening the intervention and radiation durations, and increasing the quantities of contrast substance injected into the patient.

Secondly, when new subtracted reference images are acquired or generated respectively in the case of superposition mode with subtracted reference image radioscopy, and road-map mode radioscopy, there is during the intervention a loss of information concerning the display of instruments and radio-opaque treating equipment in place due to their subtraction from the reference image. In the case of superposition mode radioscopy with non-subtracted reference image, the definition and distinction of the adjacent anatomic structures depend on the differences in radio-opacity between these structures and cause a problem when radio-opacities thereof are very close or not different enough such as in an opacified vascular, channel-like of cavity-like structure in relation with an adjacent bone structure.

Superposition mode and road-map mode radioscopies provide radio-guiding assistance based on plane reference images fixed in the reference plane, that need to be acquired or generated a priori. These reference images do not provide any information on the third dimension of the region of interest, which limits and restricts the radio-guiding assistance by these two modes of radioscopy.

An aim of the present invention is to provide an improved navigation system with respect to the above issues.

The invention provides for that purpose a method for navigation inside a region of interest, for use in a radiography unit including an X-ray source, recording means facing the source, and a support on which an object to be radiographied, including the region of interest can be positioned. The method includes the following steps:
a) acquisition of three-dimensional image data of a volume V1 of the region of interest;
b) calculation, at a time t, of a two-dimensional projection image of all or part of volume V1 and/or a sub-volume of volume V1 depending on the positions of the support, of the source and recording means, of a field of vision (FOV), a focal distance (DF) and an object distance (DO);
c) possible superposition or subtraction to the projection image and/or to the sub-volume according to a given plane section of a radioscopic image associated with the positions of the support, of the source and recording means, of the field of vision (FOV), of the focal distance (DF) and object distance (DO), at time t; and,
d) displaying on a display device an image and/or a volume resulting from step c), and/or the projection image and/or the sub-volume.

Therefore, as soon as one of the cited parameters is changed, the system automatically calculates in real time the displayed volumes and volume projection image. Consequently, the user has constant optimal display of the volume and/or projection image of the region of interest volume <<visualized>> by the radiography system, without any additional means of radiography or radioscopy, thus reducing the amount of radiation generated during the intervention, either from the resulting volume and/or resulting superposition or subtraction or fusion image, according to a plane section defined in the volume and/or on the volume projection image, of the radioscopic image of corresponding parameterization, which makes it possible for the user to optimize instrumentation guiding, control of technical intervention and evaluation of technical intervention in real time.

Optionally, the method includes at least one of the following additional features:

step b) includes the following sub-steps:

b1) reading in the storage means of the radiography device a support position (x, y, z), a source and recording means position (α, β, γ) and the values of the field of vision (FOV), focal distance (DF) and object distance (DO); and

b2) calculating the projection image and/or sub-volume according to the read parameters.

step b) includes the following sub-steps:

b1) reading in the storage means of the radiography device a support position (x, y, z) and a source and recording means position (α, β, γ);

b2) calculating sub-volume V2 of volume V1, according to these positions,

b3) reading in the storage means of the radiography device the values of field of vision (FOV), focal distance (DF) and object distance DO);

b4) calculating a corrected volume V3 of sub-volume V2 according to the field of vision (FOV), the focal distance (DF) and the object distance (DO); and

b5) optionally calculating the projected image on the basis of corrected volume V3.

the corrected volume V3 is calculated as a geometric enlargement and a scaling according to the field of vision (FOV), the focal distance (DF) and the object distance (DO).
during step b2), a projection image IP2 of sub-volume V2 is also calculated according to said positions.
during step b5), the projection image IP3 is generated by correcting the projection image IP2 according to the field of vision (FOV), the focal distance (DF) and the object distance (DO).
the calculation of correction is performed by use of an enlargement geometrical function.
the calculation of sub-volume V2 comprises the following steps:

i) determining in volume V1 an incidence axis depending on the position (α,β, γ) of the source and of the recording means relative to a reference system of the radiography device, an origin of which is an isocenter of said radiography device;

ii) determining in volume V1 a center of sub-volume V2 depending on the position (x, y, z) of the support; and

iii) calculating and reconstructing sub-volume V2 from volume V1 according to a reconstruction axis parallel to the incidence axis.

the sub-volume V2 has dimensions nx×ny×nz which are defined by an operator.
step a) includes the following sub-steps:

a1) acquiring of a set of sections through the region of interest; and

a2) reconstructing volume V1 in the form of a three-dimensional voxel matrix.

step c) includes the following sub-steps:

c1) reading the radioscopic image in the storage means of the radiography device, and

c2) superposing said image on, or subtracting said image from the projection image and/or sub-volume according to a given plane section of the radioscopic image.

The present invention also provides a radiography device, comprising an X-ray source, recording means facing said source, a support on which an object to be radiographied, containing a region of interest, can be positioned, characterized in that it comprises three-dimensional data acquisition means connected to the recording means, computing means and display means, said means being together arranged so as to perform the method according to any one of the preceding claims.

Other features and advantages of the present invention will be described hereafter:

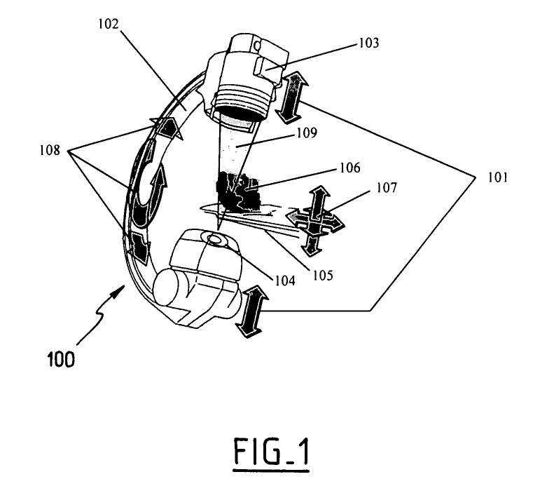

FIG. 1 illustrates the positioning of a region of interest inside a radiography device (of angiography type) according to the invention.

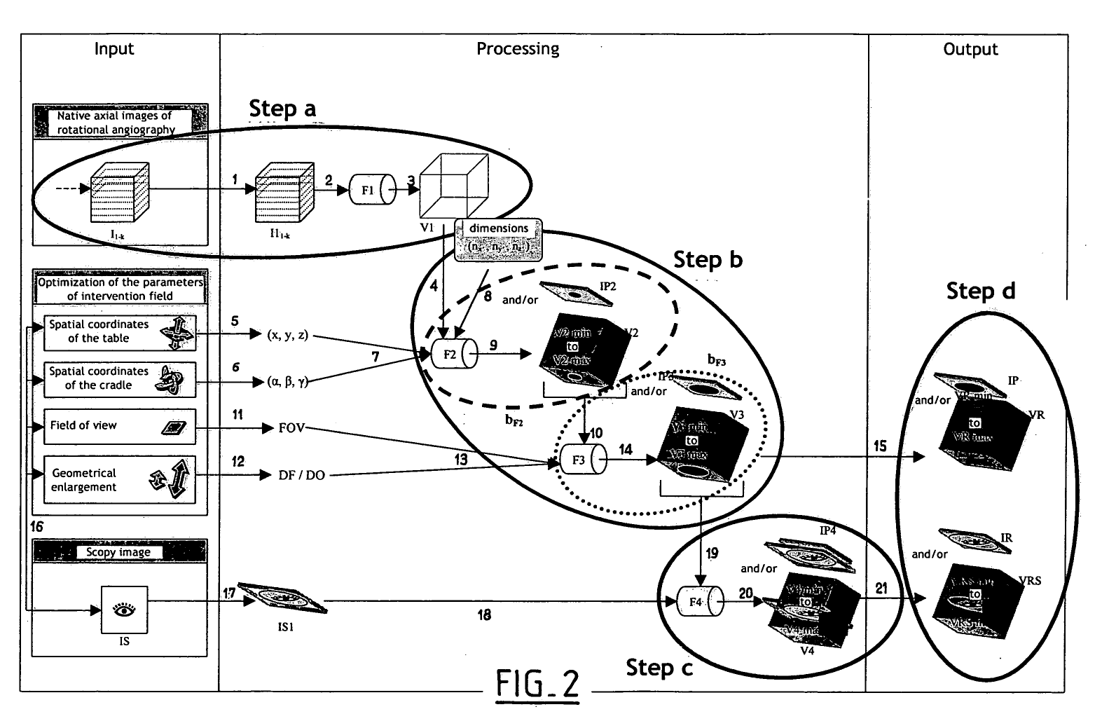

FIG. 2 is a logic diagram of the method according to the present invention.

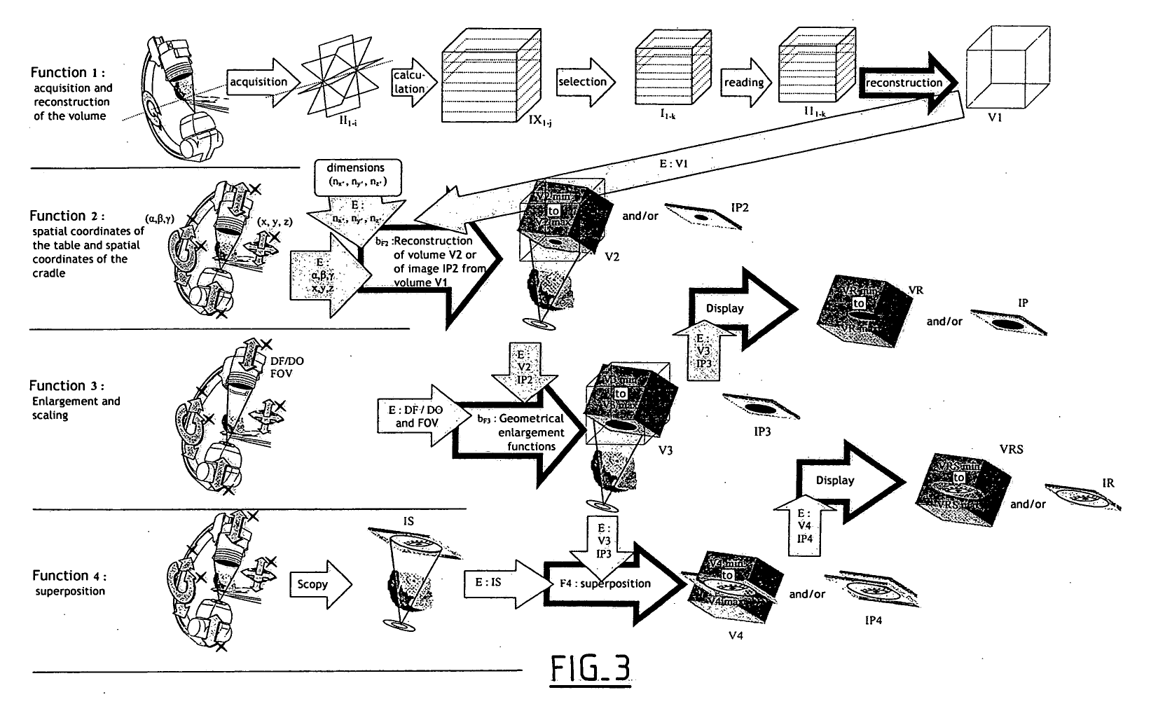

FIG. 3 is a detailed logic diagram of the various functions of FIG. 2.

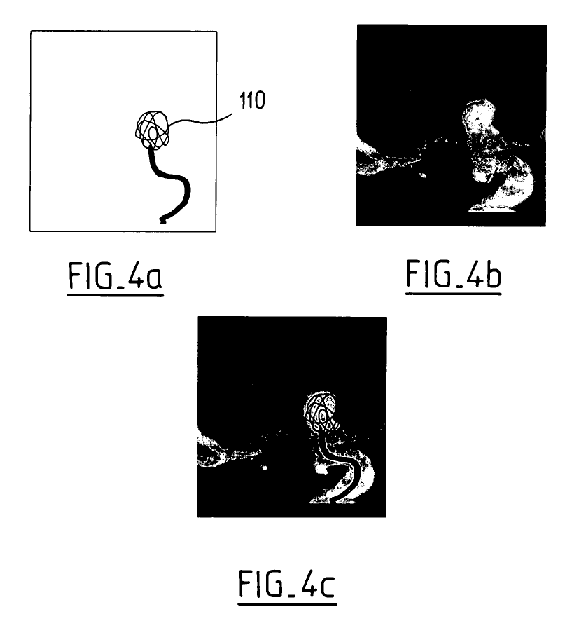

FIG. 4a,4b,4c illustrate the results of the method according to the invention.

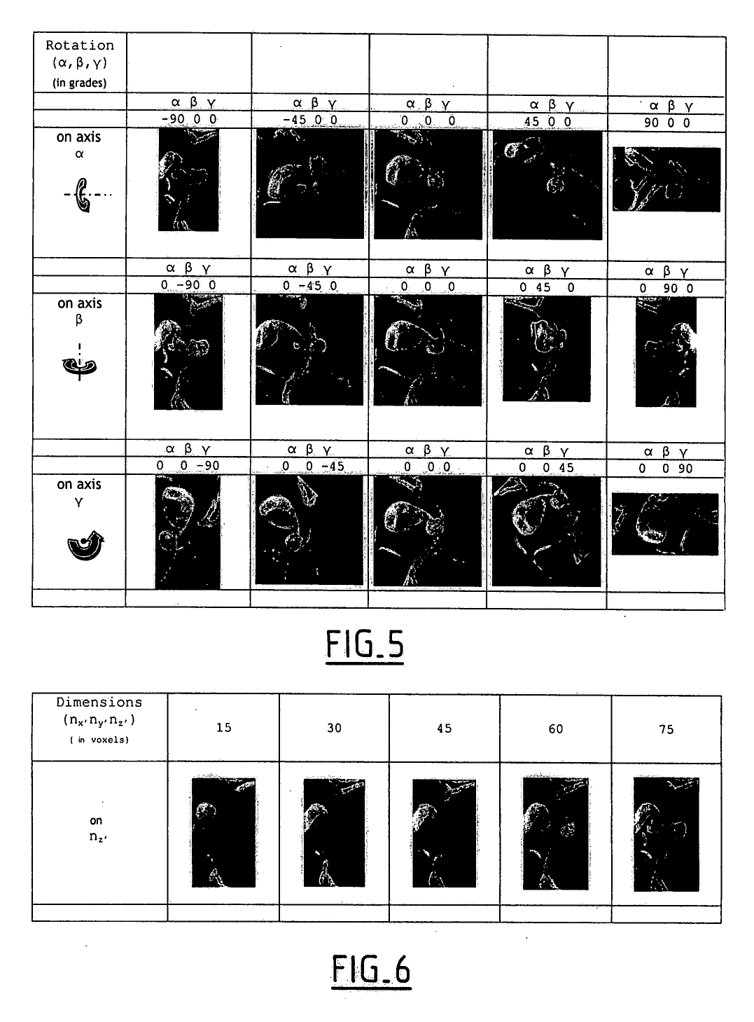

FIGS. 5 and 6 illustrate the calculation of the projection image in MIP (Maximum Intensity Projection) on the basis of the initial volume of the region of interest according to various positions of the radiography device.

Referring to FIG. 1, we shall hereinafter describe an application frame for the method of the present invention. A radiography device 100 includes a cradle 102 and a support 105, a table in this case, designed to support an object 106, in this case the head of the patient radiographied by radiography device 100 in view of an intervention at the level of a specific anatomic region, for example. Cradle 102, formed in half-circle, includes, at one end a X-ray source 104 and at the other end X-an ray sensor 103 designed for the acquisition of radiographic and radioscopic images in the region of interest positioned in an X-ray cone 109 emitted by source 104. In working position, an active surface of sensor 103 is located opposite X-ray source 104. X-ray source 104 and X-ray sensor 103 can be placed nearer of farther from each other (see arrows 101). The relative positions of X-ray source 104 and X-ray 103 are materialized by the distance between them and are represented by the focal distance parameter (DF) that the angiography device 100 constantly records in the storage means provided for this purpose (not shown). As well, the relative positions of X-ray source 104 and the region of interest of the object 106 to be radiographied are materialized by the distance between them and represented by the object distance parameter (DO) that the angiography device 100 constantly records in storage means provided for this purpose (not shown).

The field of view, the values of which are predetermined according to radiology equipment 100, is defined by a parameter (FOV) that is constantly recorded by angiography device 100 in storage means provided for this purpose (not shown).

On the other hand, cradle 102 can move according to three rotations of space as illustrated by arrows 108. This spatial position of the cradle is represented by angular coordinates (α, β, γ) constantly recorded by angiography device 100 in storage means provided for this purpose (not shown). Support table 105 can move according to three translations of space illustrated by arrows 107. As previously, the position of support table 105 is represented by rectangular coordinates (x, y, z) constantly recorded in storage means provided for this purpose (not shown).

All these parameters, rectangular coordinates (x, y, z) of table 105, angular coordinates (α, β, γ) of cradle 102, focal distance (DF), object distance (DO) and field of view (FOV) almost permanently changed by the operator during the intervention, will drive the method of this invention which shall now be described.

### Definitions:

The reference point 0 of radiography device 100 is the isocenter represented by the point of intersection of virtual lines crossing the axis of the radiogenerating tube that forms X-ray source 104, and the center of the shining amplifier including x-ray sensor 103 for two different positions of cradle 102.

The spatial coordinates of cradle 102 are determined by angular coordinates (α, β, γ). Isocenter 0 represents the position of reference point 0 of cradle 102 in radiography device 100. The origin of angular coordinates (α=0°, β=0°, γ=0°) is defined by the vertical position at 0° of right and left side inclination angle, and front and back (or cranio-caudal or caudo-cranial) longitudinal inclination of cradle 102 in relation to support table 105 designed to support object 106.

The spatial coordinates of table 105 are determined by rectangular coordinates (x, y, z). The position of reference point 0 of table 105 and reference point of rectangular coordinates (x-0, y-0, z-0) depend on the position of table 105 when the region of interest of object 106 is positioned in isocenter 0 to carry out angiography as explained hereafter. The field of view (FOV) parameter of radiography device 100 depends on the characteristics of the radiography equipment and preferentially corresponds to one of values 33, 22, 17 and 13 cm. The FOV reference value is used to carry out image acquisition by rotational angiography.

The focal distance (DF) and object distance (DO) parameters characterize length on the axis of the radiogenerating tube forming X-ray source 104 that passes through the center of the shining amplifier including x-ray sensor 103. The reference values of focal distance (DF) and object distance (DO) are those used to carry out image acquisition by rotational angiography.

With reference to FIGS. 2 and 3, the method of this invention will now be described. In FIG. 2, the “input” column of the table includes all data provided by radiography device 100, according to the invention. The method of this invention is illustrated in the processing column on FIG. 2. The output column illustrates data provided back to the user according to the invention.

Step a) of the metho, previously to the intervention itself, consists in acquiring a number of images in the region of interest and reconstruct a three-dimensional volume V1. The rotational angiography method is usually applied. This method consists in taking a series of native plane projection images of object 106 including the region of interest visualized under various incidence angles according to cradle rotation, with a view to a three-dimensional reconstruction. The region of interest to be reconstructed is positioned in the isocenter, as illustrated in FIG. 1, object 106 is then explored with a series of acquisition of angular native images II 1-i by cradle rotation in a given rotation plane in order to be visualized under various incidence angles. This is illustrated on the first two images of the first line on FIG. 3. During the acquisition of angular native images II 1-i, radiography device 100 has following parameters:
various parameters pre-defined before cradle rotation starts: frequency of image acquisition (FREQ), field of view (FOV), focal distance (DF), object distance (DF) of the region of interest of object to be radiographied from the x-ray source 104, the range of cradle 102 rotation represented by the maximum rotation angle (ANG-MAX), the rotation speed of cradle 102 as well as the rectangular coordinates (x, y, z) of support table 105 so that the region of interest of object 106 to be radiographied is positioned in the isocenter and remains in the field of visualized images during rotation of cradle 102,
variable parameters during rotation of cradle 102 for acquisition of angular coordinates (α, β, γ) of cradle 102 varying in the rotation plan.

The number of images acquired by angle degree is determined by the rotation speed of cradle 102 and image acquisition frequency (FREQ). Total number i of images acquired is determined by the number of images by angle degree and the rotation range of cradle 102 (ANG-MAX). Angular native projection images II 1-i of various incidences in the region of interest of object resulting from rotational angiography acquisition are visualized perpendicular to the rotation plane of cradle 102, under various incidences depending on the position of cradle 102 during rotation, thus making it possible to acquire images under various visual angles.

Then, in the following step, all angular native images II 1-i are changed into axial native images IX 1-j. Angular native projection images II 1-i of various incidences of object 106 including the region of interest, obtained by rotation of cradle 102, are recalculated and reconstructed in axial projection IX 1-j to obtain a series of images following a predetermined axis in view of a three-dimensional reconstruction, considering all or part of XI 1-j images after selection of a series of images I 1-k (k ranging from 1 to j) corresponding to the region of interest. These actions are directly carried out by radiography device 100. All axial native images I 1-k of rotational angiography are acquired following the inventive method. (arrow 1, FIG. 2) with the recording devices of radiography device 100 where they are stocked. The axial native images are then used as input data II 1-k (arrow 2) for a reconstruction function F1. Function F1 is used to carry out three-dimensional reconstruction to obtain a volume of the region of interest of object 106 on the basis of the input data of axial native images II 1-k. Volume V1, corresponding to the output data of function F1 (arrow 3), includes several voxels.

A voxel is the volume unit corresponding to the smallest element of a three-dimensional space, and presents individual characteristics, such as color or intensity.

Voxel stands for “volume cell element”. A three-dimensional space is divided into elementary cubes and each object is described by cubes. Volume V1 is a three-dimensional matrix of 1 voxel by h voxels by p voxels. This three-dimensional matrix representing volume V1 is the conclusion of step a) according to the present invention.

Following steps b), c) and d) are preferentially carried out pre-operatively, while the patient is operated.

The second step of the method of this invention corresponds to step b) and comprises sub-steps b F2) et F3) corresponding to F2 and F3 described hereafter. During phase b F2), the input data used by function F2 include the three-dimensional matrix of volume V1 (arrow 4) and the rectangular coordinates (x, y, z) (arrow 7), at time t, of support table 105, which are read (arrow 5) in the storage means of the rectangular coordinates of radiography device 100, illustrating the position of table 105 at time t, together with the angular coordinates (α, β, γ) (arrow 7) at time t, of cradle 102, read (arrow 6) in the storage means of radiography device 100, illustrating the position of cradle 102 at time t.

Another input datum may be provided to function F2 (arrow 8) and corresponds to dimensions (nx, ny, nz) of volume V2 calculated and reconstructed by function F2 from volume V1. Parameter nx, ny, nz are variable and determined by the operator himself. These parameters are preferably expressed in voxels and range between 1 voxel and a maximum number of voxels enabling the calculation and reconstruction of volume V2 from volume V1. Minimum volume V2min corresponds to minimum values of (nx, ny, nz) (i.e. 1 voxel) and maximum volume V2max corresponds to the maximum values of (nx, ny, nz) enabling the reconstruction of volume V2 from volume V1.

On the basis of all input data, function F2 calculates and reconstructs, from volume V1 at time t, volume V2 and possibly a projection image IP2 of volume V2 corresponding to coordinates (x, y, z) of table 105 and (α, β, γ) of cradle 102 and to dimensions (nx, ny, nz) of volume V2. When function F2 is completed, the data of volume V2 and possible projection image IP2 of volume V2 are available (volume V2 ranging from volume V2min and volume V2max corresponding to extreme values of nx, ny, nz (arrow 9). Volume V2 is reconstructed from volume V1 and parameterized at time t, by coordinates (x, y, z) of support table 105 and (α, β, γ) of cradle 102, as well as dimensions (nx, ny, nz) ranging from 1 voxel (volume V2min of one voxel reconstructed from volume V1) to maximum dimensions determining volume V2max reconstructed from volume V1.

Calculation and reconstruction of volume V2 from volume V1 are preferably carried out according to the following algorithm:
determination in volume V1 of incidence axis according to (α, β, γ) in relation to the reference system of angiography room 100 (zero point represents the isocenter) and of the position of volume V2 center according to (x, y, z) in relation to the reference system of table support 105 (zero point is determined by the position of the table during acquisition of images used to reconstruct volume V1 of object 106 region of interest, as indicated in previously mentioned definitions;
initiation by the operator or determination of dimensions nx, ,ny, nz in number of voxels of volume V2 and,
calculation and reconstruction from volume V1, of volume V2 by trilinear interpolation between voxels of a series of voxels of volume V1, with a center of dimension (nx, ny, nz) voxels according to a reconstruction axis represented by previously determined incidence axis.

Projection image IP2 is calculated by projection according to the incidence axis on a plane perpendicular to this axis, of volume V2.

Volume V2 is represented in the form of a three-dimensional matrix of nx voxels by ny voxels by nz voxels. Volume V2 and volume V2 projection image IP2 are used as input data for a function F3 used as input data for a function F3 making following phase bF3) of step b) (arrow 10). Three other parameters are used as input data (arrow 13) for function F3:
Parameter (FOV) (arrow 13), at time t, of field of view, read (arrow 11) in the storage means of this parameter of radiography device 100,
Parameter (DF) (arrow 13), at time t, of focal distance, read (arrow 12) in the storage means of this parameter of radiography device 100, and
Parameter (DO) (arrow 13), at time t, of object distance, read (arrow 12) in the storage means of this parameter of radiography device 100.

The position of the region of interest of object 106 to be radiographied in relation to x-ray source 104 and x-ray sensor 103 at time t determines the geometric enlargement parameter (DF/DO), at time t, defined by the relation between focal distance (DF) at time t, and object distance (DO) at time t.

On the basis of all input data, function F3 calculates, at time t, the geometric enlargement and the scaling of volume V2 reconstructed from volume V1, as well as projection image IP2 of volume 2. According to field of view (FOV), object distance (DO) and focal distance (DF) parameters, function F3 applies geometric enlargement function, in this case Thales geometric function, integrating the fact that the relation between a dimension in volume V2 reconstructed from volume V1 of the region of interest of object 106 or a dimension on projection image IP2 of volume V2, and the dimension in the corresponding zone of the region of interest is equal to the relation between the focal distance (DF) and the object distance (DO) of x-ray source 104 in the zone corresponding to the region of interest of object 106 where dimension is taken into account.

In output (arrow 14), function F3 provides a volume V3 corrected from volume V2 as well as a projection image IP3 of volume V3 or a projection image IP3 corrected from projection image IP2 of volume V2. Volume V3 is a volume calculated and reconstructed from volume V1 and parametered at time t by coordinates (x, y, z) of table 105, and (α, β, γ) of cradle 102, the parameters of geometric enlargement and scaling of field of view (FOV), of object distance (DO) and focal distance (DF) as well as dimensions (nx, ny, nz) ranging from 1 voxel (volume V3min of one voxel reconstructed from volume V1) to maximum dimensions determining volume V3max reconstructed from volume V1. As for volumes V1 and V2, volume V3 has the form of a three-dimensional matrix of voxels.

Once volume V3 and projection image IP3 of volume V3 are calculated, the method of this invention can transfer volume V3 and/or projection image IP3 onto display devices (arrow 15) readable at time t by the user. The user can see, at time t, on the display devices a volume VR of region of interest (volume V3 transmitted) and/or projection image IP (image IP3 transmitted) of the region of interest volume, corresponding to the relative position of support 105, of cradle 102, and the values of field of view (FOV), object distance (DO), focal distance (DF) parameters and dimensions (nx, ny, nz) at the time t. It should be noted that no radiography nor radioscopy device has been used to provide a representation of this volume and/or projection image of this volume.

During the intervention, the operator can introduce into the region of interest one or several instruments 110 (FIG. 4a) for which he wants to know exact position at time. t. The operator uses the radiography device to get a radioscopic image (IS) (arrow 16) at time t, when cradle 102 has angular coordinates (α, β, γ), support table 105 rectangular coordinates (x, y, z), sensor 103 and x-ray source 104 are positioned so as to read field of view (FOV), object distance (DO) and focal distance (DF). Radioscopic image IS1 is then read (arrow 17) at time t, on the data recording devices of radiography device 100. Data corresponding to radioscopic image IS1 are used as input data (arrow 18) during step c) for a function F4. Function F4 includes as input data: volume V3 and/or projection image IP3 of volume V3 (arrow 19) and radioscopic image IS1, read at time t in the storage means of radiography device 100.

Function F4 carries out superposition or subtraction or fusion, at time t, in volume V3 according to a defined plane section and/or on projection image IP3 of previously calculated volume V3 of radioscopic image IS1 of corresponding parameter settings (arrow 16) in relation to coordinates (x, y, z) of table 105 and (α, β, γ) of cradle 102 as well as values of field of view (FOV), object distance (DO) and focal distance (DF). At time t, function F4 superposes or subtracts in volume V3 according to a defined plane section and/or on projection image IP3 of volume V3, radioscopic image IS1, and/or calculates a projection image IP4 of volume V4 resulting from the superposition or subtraction or fusion in volume V3 according to a defined plane section of radioscopic image IS1 (projection is made in a plane parallel to the plane of radioscopic image IS1 and in a direction perpendicular to radioscopic image IS1. Function F4 provides in output (arrow 20) volume V4 and/or projection image IP4 resulting form previously described superposition or subtraction or fusion. The method of this invention can transfer volume V4 (or volume VRS) and/or projection image IP4 (or image IR) so as to display them (arrow 21) on display devices consulted, at time t, by the operator. In this way, the operator can refer to volume VRS of region of interest and/or projection image IR of region of interest volume corresponding to relative position of support 105, cradle 102 and values of field of view (FOV), object distance (DO), focal distance (DF) parameters and dimensions (nx, ny, nz) at time t. The operator knows the exact position, according to parameters predetermined at time t, of instruments 110 in the region of interest, as illustrated in FIGS. 4a to 4c.

In FIG. 4a, a radioscopic image ISD1 taken at time t is illustrated, visualizing instruments and materials 110. FIG. 4b shows the projection image IP3 of an arterial structure including intracranial aneurism, calculated as previously described, corresponding to parameters (x, y, z), (α, β, γ), (FOV), (DO), (DF) and (nx, ny, nz) associated with radioscopic image IS1 of FIG. 4a. FIG. 4c illustrates a projection image IRS resulting from the superposition carried out by function F4 during step c) when radioscopic image IS1 of FIG. 4a was superposed on projection image IP3 of FIG. 4b, illustrating the way the operator checks the positioning of his instrumentation 110 during an intervention on aneurism.

Then, at time t+δt, the operator:
either displaces his instruments 110 and wants to follow their movement with a new radioscopic image taken at time t+δt, which results in repeating, at time t+δt, previously described step c) and displaying, at time t+δt, volume VRS and/or projection image IR;
and/or modifies the relative position of cradle 102 and/or table 105, which results in repeating at time t+δt, phase bF2 of step b, and displaying volume VR and/or projection image IP. A new radioscopic image input implements step c);
and/or modifies focal distance (DF) and/or object distance (DO), which results in repeating, at time t+δt, phase bF3 of step b) and displaying, at time t+δt, volume VR and/or projection image IP. A new radioscopic image input implements step c).

In the given example, FIGS. 5 and 6 represent the result of the calculation of a projection image IP according to different positions of cradle 102. The first line of images of FIG. 5 corresponds to a variation of cradle 102 angle α to −90°, −45°, 0°, 45° and 90° while other angles β, γ are still equal to 0°. The second line of images illustrates a similar variation of angle β while α and γ are fixed to 0°. For the third line of images, α, β are fixed to 0° and γ varies. For all images, the size of initial volume V1 is: I=256 voxels by h=256 voxels by p=153 voxels.

FIG. 6 illustrates, for fixed spatial coordinates (α, β, γ) and (x, y, z), the calculation of a projection image IP according to different values of nz′ (nx′ and ny′ are unchanged), respectively 15 voxels, 30 voxels, 45 voxels, 60 voxels and 75 voxels.

In a practical and preferred manner, to validate the above-described method, the programmation language used is the Java language. It is made from the association of several software modules or plug-ins, each adding functionalities as previously described.

Preferably, they make it possible to use basic functions for processing images of any format, especially DICOM format used in radiology. Basic functions consist in reading, displaying, editing, analyzing, processing, saving and printing images. It is possible to make statistics on a pixel, or a voxel, or on a defined area. Distance and angle measures can be made, together as processing of densities and main standard imaging functions such as contrast modification, edge or median filter detection. They can also carry out geometric modifications such as enlargement, change of scale, rotation; every previous analysis and processing function can be used for any enlargement.

In addition, every function specific to the inventive method is implemented by a dedicated plug-in. Preferentially, a plug-in can calculate and reconstruct orthogonal sections in relation to a give volume or region axis.

Another plug-in calculates and reconstructs a volume, and associated projection image, by modifying the picture on every group of voxels and/or sections. This plug-in reconstructs volume according to a given axis. Volume can be turned, enlarged, or reduced. Volume interpolation is a three-linear interpolation, except from end-of-pile sections and/or end voxels where three-linear interpolation is impossible. In this case, an interpolation of the nearest neighbour is used.

Another plug-in can make a projection according to an axis, in maximum projection intensity (MIP) for example. Projection image IP3 of volume V3 can be calculated in this way.

The inventive method implements many previously described plug-ins to calculate a volume projection image. In the case of a value modification of parameters (x, y, z) of table support 105 position, or position (α, β, γ) of cradle 102, or field of view (FOV), object distance (DO) in relation to the source, focal distance (DF) or dimensions (nx, ny, nz) of studied volume, (nx, ny, nz) defined by the operator, the inventive method implements the volume reconstruction plug-in, recalculated according to the angular projection (α, β, γ) of the region of interest, then calculates enlargement and scaling in relation to the field of view (FOV) and the relation between focal distance (DF) by object distance (DO) in relation to the source, and then, with the projection plug-in, calculates volume projection image and displays projection image IP of this volume on display devices after or not superposition or subtraction or fusion of associated radioscopic image IS1.

Three-dimensional imaging acquired by rotational angiography provides better understanding of the real anatomy of a wound or anatomic structure by showing every angle required. It can be used for diagnosis. From the therapeutic point of view, its use is limited to the optimization of viewing angles, either before treatment to define a therapeutic strategy a priori, or after treatment to evaluate the therapeutic result. The implementation of reference three-dimensional imaging in per-therapy is a new concept, never used before in projection imaging, to adapt and adjust decisions and strategies, assist and control technical intervention and evaluate therapeutic results.

An implementation frame for the inventive method is described according to a projection imaging technique using an angiography device, for the investigation and endovascular treatment of an intracranial aneurism. The region of interest is represented by the corresponding intra-cranial arterial vascular structure showing intra-cranial aneurism.

In the medical domain, images used for reconstruction of the region of interest volume in three dimensions can be acquired by imaging techniques, including endo-virtual reconstruction methods:
1) projection imaging techniques such as previously described rotational angiography,
2) section imaging techniques such as computerized tomodensimetry or scanner, magnetic resonance imaging or ultra-sound imaging,
3) video imaging techniques,
4) virtual digital imaging techniques.

Images used for three-dimensional reconstruction of region of interest volume can be acquired from any previously described techniques.

Real time active images can be:
1) radioscopic for radiology and angiography techniques,
2) cinescopic for imaging techniques of computerized tomodensimetry or scanographic, by magnetic resonance, or ultrasound,
3) videoscopic for video imaging techniques such as endoscopy or coelioscopy,
4) digital for digital camera or virtual digital images.

Real time active images can be two-dimensional, stereoscopic or three-dimensional images

The imaging technique producing real time active images and the technique used for acquisition in the frame of three-dimensional reconstruction of region of interest volume can rely on one or several techniques, then requiring a positioning of the region of interest volume according to an internal or external reference system.

Display devices can include:
1) two-dimension displays providing volume projection images,
2) three-dimension simulated displays giving an impression of volume,
3) three-dimension displays (new technologies, such as holography systems) where several volumes can be mixed, added or subtracted.

During interventional radiology procedures supported with the inventive method, real time data acquisition concerning the third dimension of the studied anatomic region considered as a whole (volume and volume projection image of the region of interest) or partly by showing a hidden zone of the studied anatomic region (volume and volume projection image of a part of the region of interest), in real time (i.e. in per-procedure with almost instant response), dynamically (i.e. changing in case of modification in the parameterization of image acquisition system such as table or cradle position, values of field of view, focal distance of x-ray source from recording devices, or distance between the region of interest and x-ray source in the case of angiography device), interactively (i.e. responsive to the operator request) , under every viewing angle (i.e. corresponding to every possible incidence angle in radiography or radioscopy in the case of angiography device, for example), which, when they are superposed on or subtracted from instrument and radio-opaque equipment image data obtained by subtracted or non-subtracted active radioscopic image, provide optimization of data concerning the region of interest and the position of instruments and radio-opaque equipment in the region of interest and allow the operator to make adapted decisions in real time, in the course of the investigation or intervention concerning the definition of relevant investigation or intervention fields of view in the region of interest, investigation or intervention strategies, instrumentation guiding, technical gesture control and evaluation. Consequently, investigation or intervention safety and efficiency are optimized, intervention is shorter, the quantities of injected contrast substance and irradiation of patient and operator are reduced.

Naturally, modifications can be made within the frame of the present invention.

1. A method for navigation inside a region of interest, for use in a radiography unit (100) including an X-ray source (104), recording means (103) facing the source, and a support (105) on which an object (106) to be radiographied, containing the region of interest, can be positioned, the method comprising the following steps:
a) acquiring three-dimensional image data of a volume V1 of the region of interest;
b) calculating, at a time t, a two-dimensional projection image (IP, IP2, IP3) of all or part of volume V1 and/or a sub-volume (V2, V3, VR) of said volume V1 according to the position of the support (105), the position of the source (104) and recording means (103), a field of vision (FOV), a focal distance (DF) and an object distance (DO);
c) optionally superposing to, or subtracting from to the projection image (IP, IP3) and/or to the sub-volume (V3, VR), according to a given plane section, a radioscopic image (IS1) associated with the positions of the support (105), of the source (104) and recording means (103), of the field of vision (FOV), of the focal distance (DF) and object distance (DO), at time t; and
d) displaying on a display device an image (IR) and/or a volume (VRS) resulting from step c), and/or the projection image (IP, IP2, IP3) and/or the sub-volume (V2, V3, VR).

2. A method according to claim 1, characterized in that step b) includes the following sub-steps:
b1) reading in the storage means of the radiography device a support position (x, y, z), a source and recording means position (α, β, γ) and the values of the field of vision (FOV), focal distance (DF) and object distance (DO); and
b2) calculating the projection image (IP, IP3) and/or sub-volume (V3, VR) according to the read parameters.

3. A method according to any one of claims 1 and 2, characterized in that step b) includes the following sub-steps:
b1) reading in the storage means of the radiography device a support position (x, y, z) and a source and recording means position (α, β, γ);
b2) calculating sub-volume V2 of volume V1, according to these positions,
b3) reading in the storage means of the radiography device the values of field of vision (FOV), focal distance (DF) and object distance DO);
b4) calculating a corrected volume V3 of sub-volume V2 according to the field of vision (FOV), the focal distance (DF) and the object distance (DO); and
b5) optionally calculating the projected image (IP, IP3) on the basis of corrected volume V3.

4. A method according to claim 3, characterized in that the corrected volume V3 is calculated as a geometric enlargement and a scaling according to the field of vision (FOV), the focal distance (DF) and the object distance (DO).

5. A method according to claim 3, characterized in that, during step b2), a projection image (IP2) of sub-volume V2 is also calculated according to said positions.

6. A method according to claim 5, characterized in that, during step b5), the projection image (IP, IP3) is generated by correcting the projection image (IP2) according to the field of vision (FOV), the focal distance (DF) and the object distance (DO).

7. A method according to claim 4 or 6, characterized in that the calculation of correction is performed by use of an enlargement geometrical function.

8. A method according to any one of claims from 3 to 7, characterized in that the calculation of sub-volume V2 comprises the following steps:
i) determining in volume V1 an incidence axis depending on the position (α, β, γ) of the source (104) and of the recording means (103) relative to a reference system of the radiography device, an origin of which is an isocenter of said radiography device;
ii) determining in volume V1 a center of sub-volume V2 depending on the position (x, y, z) of support (105); and
iii) calculating and reconstructing sub-volume V2 from volume V1 according to a reconstruction axis parallel to the incidence axis.

9. A method according to any one of claims 3 to 8, characterized in that the sub-volume V2 has dimensions nx×ny×nz which are defined by an operator.

10. A method according to any one of the preceding claims, characterized in that step a) includes the following sub-steps:
a1) acquiring of a set of sections through the region of interest; and
a2) reconstructing volume V1 in the form of a three-dimensional voxel matrix.

11. A method according to any one of claims 1 to 10, characterized in that step c) includes the following sub-steps:
c1) reading the radioscopic image IS1 in the storage means of the radiography device, and
c2) superposing said image on, or subtracting said image from the projection image (IP, IP3) and/or sub-volume (V3, VR) according to a given plane section of the radioscopic image IS1.

12. A radiography device, comprising an X-ray source, recording means facing said source, a support on which an object to be radiographied, containing a region of interest, can be positioned, characterized in that it comprises three-dimensional data acquisition means connected to the recording means, computing means and display means, said means being together arranged so as to perform the method according to any one of the preceding claims.
